(19) United States
(12) Patent Application Publication
(10) Pub. No.: US 2024/0188823 A1
(43) Pub. Date: Jun. 13, 2024

(54) INTRAOCULAR PRESSURE DETECTING DEVICE AND DETECING METHOD THEREOF

(71) Applicant: Industrial Technology Research Institute, Hsinchu (TW)

(72) Inventors: De-Yi Chiou, New Taipei City (TW); Chi-Shen Chang, Hsinchu County (TW); Da-Wen Lu, Taipei City (TW)

(73) Assignee: Industrial Technology Research Institute, Hsinchu (TW)

(21) Appl. No.: 18/079,006

(22) Filed: Dec. 12, 2022

(51) Int. Cl. A61B 3/16; A61B 3/00

(52) U.S. Cl. CPC A61B 3/16; A61B 3/0025

(57) ABSTRACT

An intraocular pressure detecting device includes the following elements. A force-applying element is adapted to apply a force to a target surface on a cornea of an eyeball in a direction, so that the target surface is deformed. A force-sensing element, coupled to the force-applying element, is adapted to sense the force applied by the force-applying element in the direction. A displacement-sensing element, coupled to the force-applying element, is adapted to sense a displacement of the force-applying element in the direction. A processing element is electrically connected to the force-sensing element and the displacement-sensing element to obtain a relationship curve between applied force and displacement. In particular, the processing element analyzes the relationship curve to obtain a characteristic critical point, and obtains an intraocular pressure value of the eyeball according to the applied force corresponding to the characteristic critical point.

### TECHNICAL FIELD

The present application relates to an intraocular pressure detecting device and an intraocular pressure detecting method.

### BACKGROUND

In order to maintain the elasticity and visual function of the eyeball, the intraocular pressure must be maintained within a certain range. The level of intraocular pressure is related to the production and elimination of aqueous humor in the eyeball. The aqueous humor is produced by the ciliary process of the ciliary body in the posterior chamber, and flows through the pupil to the anterior chamber, then flows from the trabecular meshwork in the corner through Schlemm's canal or through the interstitial space of the uvea and recycled into the blood through veins. Aqueous humor supplies oxygen and nutrients to the tissues in the front portion of the eye and takes away the metabolic waste thereof. The balance between production and discharge of aqueous humor determines the level of intraocular pressure. If there is too much aqueous humor or the drainage path is blocked, the intraocular pressure is increased, and the high intraocular pressure compresses the optic nerve and causes damage to the optic nerve function, resulting in visual field defect and decreased vision to form glaucoma.

Excessive intraocular pressure is a high-risk group for glaucoma, but high intraocular pressure does not necessarily cause glaucoma, and people with normal intraocular pressure also have the possibility of suffering from glaucoma (normal tension glaucoma). Clinically, the intraocular pressure of normal people is in the range of 10 mmHg to 21 mmHg. The measurement of intraocular pressure is an important factor in controlling the progression of glaucoma, but the day and night fluctuations of intraocular pressure vary from person to person. Generally speaking, the intraocular pressure of normal people fluctuates by 2 mmHg to 6 mmHg, and the intraocular pressure of glaucoma patients fluctuate more, even greater than 10 mmHg.

Therefore, the development of an instrument suitable for patients to detect intraocular pressure at home to effectively monitor and control intraocular pressure from elevating and further damaging vision is helpful for the clinical monitoring and treatment of early glaucoma.

### SUMMARY

The present application provides an intraocular pressure measuring device including a force-applying element, a force-sensing element, a displacement-sensing element, and a processing unit. The force-applying element is adapted to apply a force to a target surface on a cornea of an eyeball in a direction, so that the target surface is deformed. The force-sensing element, coupled to the force-applying element, is adapted to sense the applied force of the force-applying element in the direction. The displacement-sensing element, coupled to the force-applying element, is adapted to sense a displacement of the force-applying element in the direction. The processing element is electrically connected to the force-sensing element and the displacement-sensing element to obtain a relationship curve between the applied force and the displacement. In particular, the processing element analyzes the relationship curve between the applied force and the displacement to obtain a characteristic critical point, and obtains an intraocular pressure value of the eyeball according to the applied force corresponding to the characteristic critical point.

The present application provides an intraocular pressure detecting method including the steps of: applying a force to a target surface on a cornea of an eyeball in a direction, so that the target surface is deformed; sensing the applied force in the direction; sensing a displacement in the direction; obtaining a relationship curve between the applied force and the displacement; analyzing the relationship curve between the applied force and the displacement to obtain a characteristic critical point; and obtaining an intraocular pressure value of the eyeball according to the applied force corresponding to the characteristic critical point.

The present application further provides an intraocular pressure detecting method, including the steps of: applying a force to a cornea on an eyeball and an eyelid covering the cornea in a direction, so that the eyelid and the cornea are deformed; sensing the applied force in the direction; sensing a displacement in the direction; obtaining a relationship curve between the applied force and the displacement; analyzing the relationship curve between the applied force and the displacement to obtain a boundary point of the relationship curve between the applied force and the displacement, wherein an applied force corresponding to the boundary point is a first applied force, the boundary point divides the relationship curve between the applied force and the displacement into a first curve portion and a second curve portion, the first curve portion is between an origin of the relationship curve between the applied force and the displacement to the boundary point, and the second curve portion is the remaining relationship curve between the applied force and the displacement; analyzing the second curve portion to obtain a characteristic critical point, wherein the applied force corresponding to the characteristic critical point is a second applied force; and calculating an intraocular pressure value of the eyeball according to a difference between the second applied force and the first applied force.

### BRIEF DESCRIPTION OF THE DRAWINGS

FIG. 1 is a schematic diagram of an intraocular pressure detecting device according to an embodiment of the present application.

FIG. 2 is a relationship graph between applied force and displacement of an intraocular pressure detecting device according to an embodiment of the present application.

FIG. 3A to FIG. 3E are schematic diagrams of an intraocular pressure detecting method according to an embodiment of the present application.

FIG. 4 is a schematic diagram of an intraocular pressure testing platform according to an embodiment of the present application.

FIG. 5A is a relationship graph between applied force and displacement of an intraocular pressure detecting device according to an embodiment of the present application.

FIG. 5B is a relationship graph between slope and displacement of an applied force-displacement relationship curve of an intraocular pressure detecting device according to an embodiment of the present application.

FIG. 6 is a schematic diagram of an intraocular pressure detecting device according to an embodiment of the present application.

FIG. 7 is a relationship graph between applied force and displacement of an intraocular pressure detecting device according to an embodiment of the present application.

FIG. 8 is a relationship graph between applied force and displacement of an intraocular pressure detecting device according to an embodiment of the present application.

### DETAILED DESCRIPTION OF DISCLOSED EMBODIMENTS

Embodiments are provided hereinafter and described in detail with reference to figures. However, the embodiments provided are not intended to limit the scope of the present application. In addition, the element sizes in the drawings are drawn for convenience of description and do not represent actual element size ratios. To facilitate understanding, similar elements in the following are described with the same reference numerals.

Different examples in the description of embodiments of the present application may adopt repeated reference numerals and/or terms. These repeated numerals or terms are intended to simplify and clarify and are not intended to limit the relationship of each embodiment and/or external structures. Furthermore, if the disclosure of the present specification describes forming a first feature on or above a second feature, it includes an embodiment in which the formed first feature is in direct contact with the second feature and also includes an embodiment in which additional features are formed between the first feature and the second feature such that the first feature and the second feature may not be in direct contact.

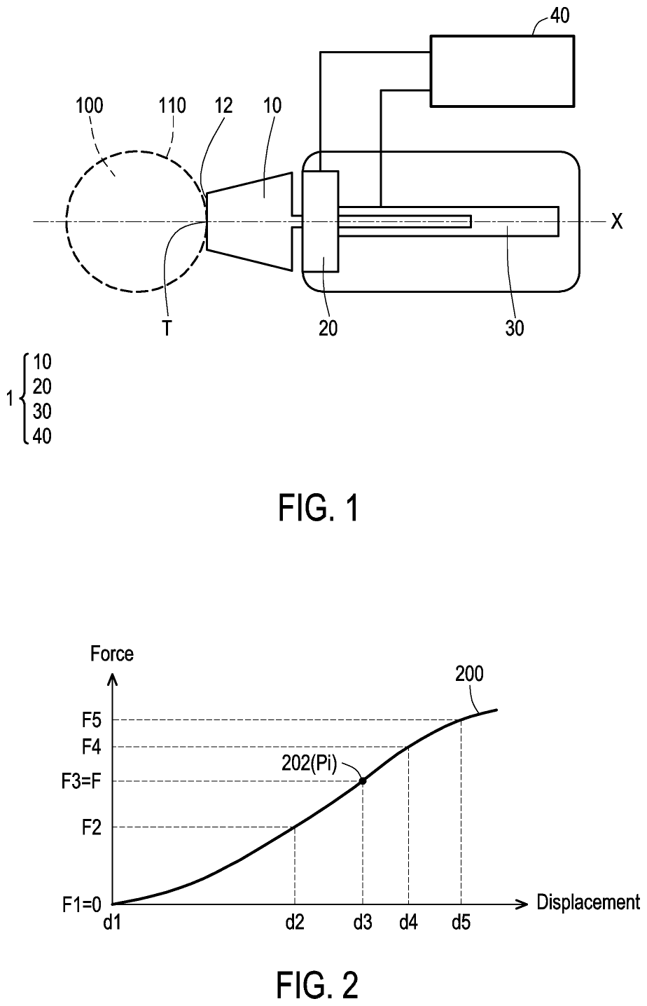

FIG. 1 is a schematic diagram of an intraocular pressure detecting device according to an embodiment of the present application. As shown in FIG. 1, an intraocular pressure detecting device 1 includes: a force-applying element 10, a force-sensing element 20, a displacement-sensing element 30, and a processing element 40.

The force-applying element 10 is adapted to apply a force F to a target surface T on a cornea 110 of an eyeball 100 in a direction X, so that the target surface T is deformed, and the aqueous humor pressure (intraocular pressure) in the cornea 110 also exerts the same amount of pressure on the surface 12 of the force-applying element 10, and the intraocular pressure is detected based on this principle. According to some embodiments, the front end of the force-applying element 10 has the surface 12 having an area of A and adapted to be in contact with the target surface T. According to some embodiments, the material of the force-applying element 10 may be a rigid body of metal or non-metal (such as polymer material), and the present application is not limited thereto. In some embodiments, the surface 12 of the front end of the force-applying element 10 is circular in shape, and the diameter thereof is 3.06 mm. If the aqueous humor pressure (intraocular pressure) in the cornea 110 is 10 mmHg, when the surface 12 of the force-applying element 10 just applanates the cornea 110, the external force needed by the force-applying element 10 is 1 g. If the aqueous humor pressure (intraocular pressure) in the cornea 110 is 20 mmHg, when the surface 12 of the force-applying element 10 just applanates the cornea 110, the external force needed by the force-applying element 10 is 2 g, and so on. In some other embodiments, when the diameter of the surface 12 is 6.12 mm, if the aqueous humor pressure (intraocular pressure) in the cornea 110 is 10 mmHg, when the surface 12 of the force-applying element 10 just applanates the cornea 110, the external force needed by the force-applying element 10 is 4 g. If the aqueous humor pressure (intraocular pressure) in the cornea is 20 mmHg, when the surface 12 of the force-applying element 10 just applanates the cornea 110, the external force needed by the force-applying element 10 is 8 g, and so on. The above description is based on basic physical principles, and the pressure is equal to total force divided by area. Therefore, when the surface 12 of the force-applying element 10 just applanates the cornea 110, the standard intraocular pressure measurement results are obtained by dividing the external force needed by the force-applying element 10 by the area value of the surface 12 of the force-applying element 10.

The force-sensing element 20, coupled to the force-applying element 10, is adapted to sense the applied force F of the force-applying element 10 in the direction X. According to some embodiments, the force-sensing element 20 may be a piezoelectric force-sensing element utilizing the piezoelectric effect to sense the magnitude of external force, or a strain-type force-sensing element measuring the magnitude of external force by measuring applied stress. According to some embodiments, the precision of the force-sensing element 20 is less than 0.05 g, so as to reduce the error of measuring intraocular pressure, but the present application is not limited thereto. According to some embodiments, the sensing range of the force-sensing element 20 is 0 g to 100 g, but the present application is not limited thereto. Moreover, in other embodiments, the force-sensing element 20 may be directly disposed on the surface of the force-applying element 10. Therefore, when the force-applying element 10 applies the force F to the target surface T of the cornea 110, the force F is applied to the cornea 110 by the force-sensing element 20, and the value of the applied force F is sensed by the force-sensing element 20 at the same time.

The displacement-sensing element 30, coupled to the force-applying element 10, is adapted to sense a displacement d of the force-applying element 10 in the direction X, that is, the deformation distance of the target surface T of the cornea 110 in the direction X. According to some embodiments, the displacement-sensing element 30 is a micro-displacement gauge, and the precision of the displacement-sensing element 30 is less than 0.01 mm to reduce the error of the force application distance, but the present application is not limited thereto.

The processing element 40 is electrically connected to the force-sensing element 20 and the displacement-sensing element 30 to obtain a relationship curve between the applied force F of the force-applying element 10 and the displacement d of the force-applying element 10. The processing element 40 analyzes the relationship curve between the applied force F and the displacement d to obtain a characteristic critical point Pᵢ, and obtains an intraocular pressure value of the eyeball 100 according to the applied force F corresponding to the characteristic critical point Pᵢ.

When performing intraocular pressure detection, the force-applying element 10 of the intraocular pressure detecting device 1 applies a force F on the target surface T on the cornea 110 of the eyeball 100 in the direction X, so that the target surface T is deformed. During the measurement process, the applied force F in the direction X is sensed by the force-sensing element 20, and the displacement d in the direction X is sensed by the displacement-sensing element 30. The processing unit 40 receives the sensing results from the force-sensing unit 20 and the displacement-sensing unit 30 to obtain the relationship curve between the applied force F and the displacement d. Next, the processing unit 40 further analyzes the relationship curve between the applied force F and the displacement d to obtain the characteristic critical point Pᵢ (details are described later). Subsequently, the processing unit 40 calculates the intraocular pressure value of the eyeball 100 according to the applied force F corresponding to the characteristic critical point Pᵢ.

In some embodiments, when the force-applying element 10 applies the force F on the target surface T of the cornea 110, the processing unit 40 continuously reads the force F of the force-applying element 10 and the displacement d of the force-applying element 10 respectively detected by the force-sensing element 20 and the displacement-sensing element 30 at a fixed time interval or an irregular time interval. In some other embodiments, when the force-applying element 10 applies the force F on the target surface T of the cornea 110, after the processing element 40 detects a fixed displacement distance or a non-fixed displacement distance at the displacement-sensing element 30, the processing element 40 then execute the action of reading the applied force F of the force-applying element 10 and the displacement d of the force-applying element 10 detected by the force-sensing element 20 and the displacement-sensing element 30. This design may avoid misjudgment caused by misoperation. According to some embodiments, the fixed displacement distance may be 0.01 mm or other values, and the present application is not limited thereto. When the processing element 40 reads more data on the applied force F of the force-applying element 10 and the displacement d of the force-applying element 10, the error of the relationship curve between the applied force F and the displacement d may be reduced. In turn, the precision of data processing is improved and measurement errors are reduced. According to some embodiments, the processing element 40 may include a central processing unit (CPU) for processing data and computer-readable instructions, and a memory for storing data and instructions. The memory may include volatile random-access memory (RAM), non-volatile read-only memory (ROM), and/or other types of memory. A data storage component may also be included for storing data and controller/processor executable instructions. The data storage component may include one or a plurality of non-volatile solid-state memory devices (such as flash memory, read-only memory (ROM), magnetoresistive RAM (MRAM), ferroelectric RAM (FRAM), phase change memory, etc.)

In traditional Goldmann Applanation Tonometry (GAT), before intraocular pressure is measured, the patient's eyes need to be anesthetized and luciferin is instilled in the eyes. Next, the patient sits in front of a slit lamp, puts his head on the chin rest, and under the illumination of the slit lamp, touches the pressure-measuring head of the tonometer inserted on the slit lamp to the cornea. The image of the indenter is observed until the cornea is completely applanated by the indenter, that is, the applanated area of the cornea is exactly equal to the surface area of the indenter. Next, the total applied force of the tonometer is divided by the surface area of the indenter to obtain the intraocular pressure value at this time.

According to the principles of Goldmann Applanation Tonometry (GAT), it may be known that the pressure value when the target surface of the cornea is just completely applanated is the intraocular pressure. Therefore, corresponding to the intraocular pressure detecting device 1 of the present application, in the process of detecting variation in the applied force F and the displacement d, the degree of applanation of the cornea 110 is evaluated by defining the characteristic critical point Pᵢ. That is, the applied force F when the target surface T of the force-applying element 10 and the cornea 110 is just completely applanated is found and converted into intraocular pressure. In addition, the exact applanation of the target surface T of the cornea 110 means that the applanated area of the target surface T is equal to an area A of the surface 12 of the force-applying element 10. The characteristic critical point Pᵢ obtained by measuring the relationship curve between the applied force F and the displacement d may replace the previous method of determining intraocular pressure by using slit lamps, fluorescent agents, and optical prisms, etc. In addition, if the intraocular pressure is measured through the eyelids, the above optical methods in the prior art are all not applicable. Therefore, the present application may also improve the intraocular pressure measurement method (detailed later) by measuring the characteristic critical point obtained from the relationship curve between the applied force F and the displacement d, so as to increase the convenience and practicability of the intraocular pressure measurement.

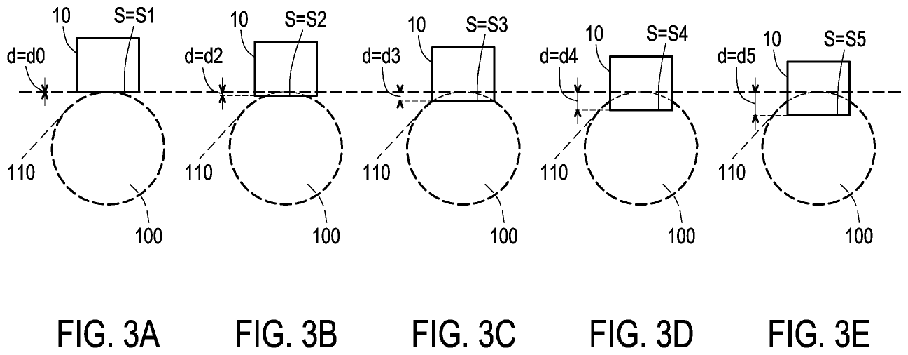

FIG. 2 is a relationship graph between applied force and displacement of an intraocular pressure detecting device according to an embodiment of the present application. FIG. 3A to FIG. 3E are schematic diagrams of an intraocular pressure detecting method according to an embodiment of the present application. Please refer to FIG. 1, FIG. 2, and FIG. 3A to FIG. 3E at the same time.

As shown in FIG. 3A, at the initial position, the surface of the force-applying element 10 is in contact with the cornea 110, and the force-applying element 10 does not apply force to the cornea 110. The position of the force-applying element 10 at this time is defined as the origin. Therefore, it is defined that the applied force F=F1=0 of the force-application element 10 at this time, and the displacement d=d1=0 of the force-application element 10. A contact area S=S1=0 of the force-applying element 10 with the target surface T of the cornea 110.

As shown in FIG. 3B, the force-applying element 10 exerts the force of F=F2 on the cornea 110. The cornea 110 is deformed after receiving the force F2, and the force-applying element 10 is displaced d=d2 in the direction X. At this time, the contact area S=S2 between the force-applying element 10 and the target surface T of the cornea 110. At this time, the contact area S2 of the target surface T is less than the area A of the surface 12 of the force-applying element 10, that is, S=S2<A.

As shown in FIG. 3C, the force-applying element 10 applies the force of F=F3 on the cornea 110. The cornea 110 is deformed after receiving the force F3, and the force-applying element 10 is further displaced d=d3 in the direction X. At this time, the contact area S=S3 between the force-applying element 10 and the target surface T of the cornea 110. At this time, the contact area S3 of the target surface T is equal to the area A of the surface 12 of the force-applying element 10, that is, S=S3=A. At this time, the degree of applanation of the cornea 110 is regarded as just completely applanated, which may be defined by the characteristic critical point of a relationship curve 200 between the applied force F and the displacement d. The applied force F=F3 at this time is exactly the force applied by the force-applying element 10 to completely applanate the cornea 110. This applied force F=F3 is converted into intraocular pressure, and according to the above Goldmann Applanation Tonometry (GAT) principle, the pressure value at this time P=F3/A is the intraocular pressure.

As shown in FIG. 3D, the force-applying element 10 exerts the force of F=F4 on the cornea 110. After the cornea 110 is deformed by the applied force F4, the force-applying element 10 continues to be displaced d=d4 in the direction X, and the cornea 110 continues to be depressed in the direction X. However, at this time, the contact area S=S4 between the force-applying element 10 and the target surface T of the cornea 110, and the contact area S4 is not increased, but is equal to the area A of the surface 12 of the force-applying element 10. At this time, the contact area S4 is equal to the area A of the surface 12 of the force-applying element 10, that is, S=S4=A.

As shown in FIG. 3E, the force-applying element 10 exerts the force of F=F5 on the cornea 110. At this time, the cornea 110 is deformed after receiving the force F5, the force-applying element 10 continues to be displaced d=d5 in the direction X, and the cornea 110 continues to be depressed along the direction X. Similar to the situation of FIG. 3D, the contact area S=S5 between the force-applying element 10 and the target surface T of the cornea 110 at this time, and the contact area S5 is not increased, but is equal to the area A of the surface 12 of the force-applying element 10. At this time, the contact area S5 is equal to the area A of the surface 12 of the force-applying element 10, that is, S=S5=A.

As shown in FIG. 3A to FIG. 3E, by applying the force F to the cornea 110 by the force-applying element 10, the relationship curve 200 between the applied force F and the displacement d as shown in FIG. 2 may be obtained. However, since the actual measurement process is performed continuously, there is no way to clearly know the degree of applanation of the cornea 110, and there is also no way to know when the situation that the cornea 110 is just completely applanated as shown in FIG. 3C and the contact area S3 of the target surface T is equal to the area A of the surface 12 of the force-applying element 10 occurs. By observing the relationship curve 200, it may be found that when the force F is initially applied to the cornea 110 by the force-applying element 10, the applied force F and the square of the displacement d are substantially in a proportional curve relationship, the contact area S of the target surface T is less than the area A of the surface 12 of the force-applying element 10 (corresponding to FIG. 3A to FIG. 3C), and the displacement d is caused by the deformation of the cornea 110. However, after the cornea 110 is completely applanated by the force-applying element 10, the contact area S of the target surface T is still equal to the area A of the surface 12 of the force-applying element 10, but the eyeball 100 starts to be deformed in reverse (corresponding to FIG. 3C to FIG. 3E), and the displacement d at this time is caused by the deformation of the eyeball 100 instead.

Accordingly, the characteristic critical point Pᵢ is defined on the relationship curve 200. The applied force F corresponding to the characteristic critical point Pᵢ represents the applied force F when the cornea 110 is just completely applanated and the contact area S of the target surface T is equal to the area A of the surface 12 of the force-applying element 10. It may be known according to the variation of the displacement d of the cornea 110 just before and after complete applanation in the relationship curve 200 that the characteristic critical point Pᵢ is an inflection point 202 of the relationship curve 200. The inflection point 202 of the relationship curve 200 may be obtained by numerically analyzing the relationship curve 200 by the processing unit 40.

Mathematically, the inflection point falls at the position where the second differential of the curve function is zero, or where the first differential of the curve function is an extreme value (maximum or minimum value). Therefore, when the processing element 40 analyzes the relationship curve 200 between the applied force F and the displacement d, in order to obtain the inflection point 202 of the relationship curve 200 as the characteristic critical point Pᵢ, the fitting function of the relationship curve 200 may be obtained using a curve fitting operation, and the fitting function may be differentiated to obtain the inflection point 202 of the relationship curve 200.

In some embodiments, the fitting function may be a polynomial function. According to some embodiments, the power of the highest order term of the polynomial function is greater than or equal to three.

In some embodiments, if the fitting function is ƒ(x)=ax³+bx²+cx+d, then the first differential and the second differential of the fitting function f(x) are respectively

First differential: ƒ′(x)=3ax²+2bx+c.

Second differential: ƒ″(x)=6ax+2b.

Since the inflection point of the fitting function f(x) falls at the position where the second differential is zero, the displacement d3 corresponding to the inflection point 202 is

$$d 3 = - \frac{b}{3 a} .$$

Therefore, the inflection point 202 of the relationship curve 200 may be obtained by the curve fitting operation, and used as the characteristic critical point Pᵢ. In addition, since the contact area S is equal to the area A of the surface 12 of the force-applying element 10, the actual intraocular pressure may be obtained by dividing the applied force F by the area A of the surface 12 of the force-applying element 10. Specifically, the processing element 40 may obtain the applied force F3 corresponding to the inflection point 202 from the relationship curve 200 according to the displacement d3 corresponding to the inflection point 202 (the characteristic critical point Pᵢ), and then obtain an intraocular pressure P, namely

$$P = \frac{F}{A} ,$$

In particular, P is the intraocular pressure, F is the force applied by the force-applying element 10, and A is the area of the force-applying element 10.

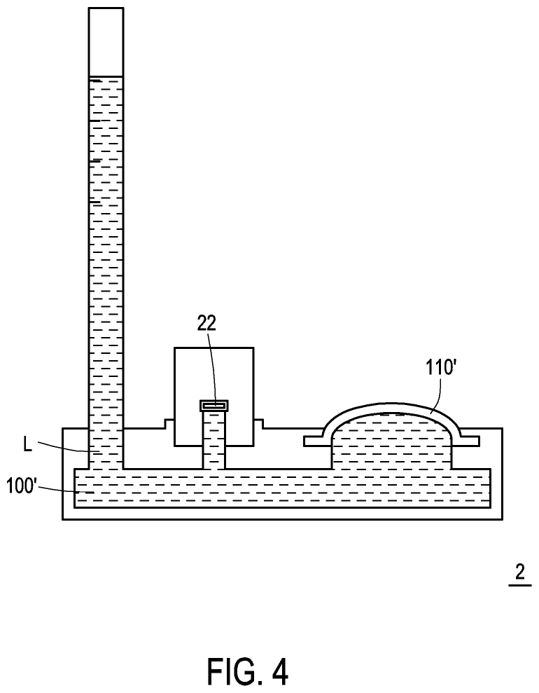

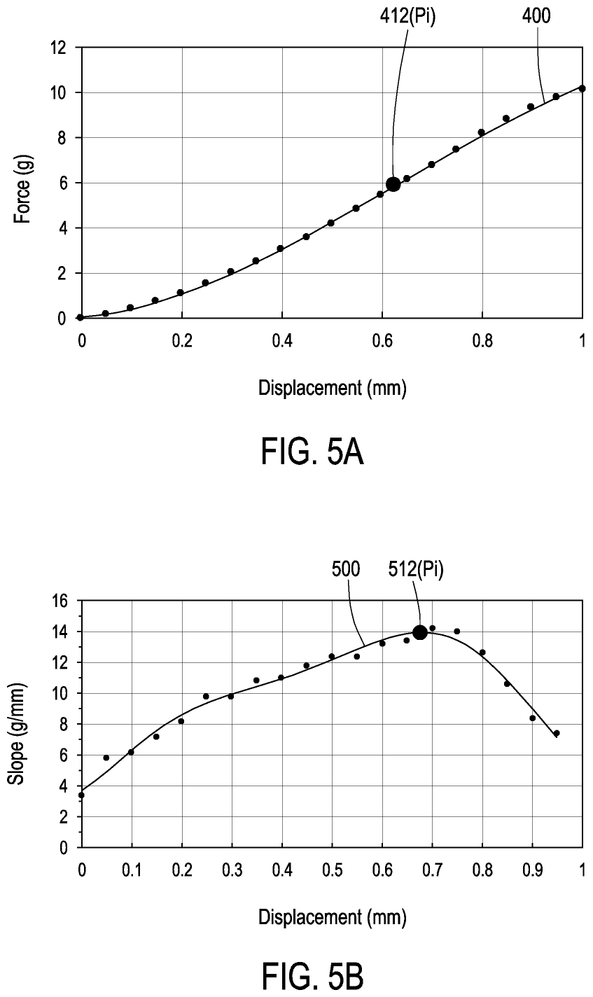

FIG. 4 is a schematic diagram of an intraocular pressure testing platform according to an embodiment of the present application. FIG. 5A is a relationship graph between applied force and displacement of an intraocular pressure detecting device according to an embodiment of the present application. Please refer to FIG. 4 first. In the present embodiment, an intraocular pressure test platform 2 is designed, having a connecting tube 100′, and the connecting tube 100′ contains a solution. An open end of the connecting tube 22 is provided with a hydraulic sensor 22, and the eyeball 100 with a specific intraocular pressure may be simulated by controlling the amount of liquid injected into the connecting tube 110′ and sensing the hydraulic pressure of a liquid level L by the hydraulic sensor 22. Moreover, at another open end of the connecting tube 100′, an artificial cornea 110′ is attached at the position of the liquid level surface L, so as to simulate the cornea 110 of the present application. Therefore, in an experimental example, the artificial cornea 110′ having a thickness of 0.5 mm, a transverse diameter of 12 mm, and a vertical diameter of 11 mm is provided, and an appropriate amount of solution is injected into the connecting tube 100′ to make the hydraulic pressure detected by the hydraulic sensor 22 reach 15 mmHg, thereby simulating the state of the eyeball 100 having an intraocular pressure of 15 mmHg. The force F is applied to the artificial cornea 110′ by the intraocular pressure detecting device 1, and via the sensing of the force-sensing element 20 and the displacement-sensing element 30, a relationship curve 400 between applied force and displacement as shown in FIG. 5A is obtained by the analysis of the processing element 40.

According to some embodiments, when the curve fitting operation is performed on the relationship curve 400 between applied force and displacement, the fitting function f(x) may be a polynomial function, wherein the power of the highest order term of the polynomial function is greater than or equal to three, but the present application is not limited thereto. As shown in FIG. 5A, in the present embodiment, the fitting function f(x) of the relationship curve 400 is a cubic polynomial, and the fitting function f(x)=−8.9391x³+17.041x²+2.1108x+0.0556.

The processing unit 40 calculates and obtains an inflection point 412 of the relationship curve 400 according to the fitting function f(x), and uses the inflection point 412 as the characteristic critical point Pᵢ. According to the fitting function f(x), the displacement corresponding to the inflection point 412 is d=0.635 mm, and the applied force is f(0.635)=5.978 g. Since the pressing plane of the force-applying element 10 is circular with a diameter of 6.12 mm, after conversion, it is equivalent to a pressure of 14.945 mmHg, which is similar to the intraocular pressure of 15 mmHg set by the artificial cornea 110′.

Therefore, as shown in FIG. 5A, the relationship curve 400 between the applied force F and the displacement d is measured by the intraocular pressure detecting device 1, and the inflection point 412 of the relationship curve 400 is obtained as the characteristic critical point Pᵢ by a curve fitting operation. Then, the intraocular pressure value is obtained from the characteristic critical point Pᵢ.

FIG. 5B is a schematic diagram of the relationship between slope and displacement of an applied force-displacement relationship curve of an intraocular pressure detecting device according to an embodiment of the present application. Please refer to FIG. 5A and FIG. 5B at the same time. In the present embodiment, the force F is applied to the artificial cornea 110′ by the intraocular pressure detecting device 1, and via the sensing of the force-sensing element 20 and the displacement-sensing element 30, the relationship curve 400 between applied force and displacement as shown in FIG. 5A is obtained by the analysis of the processing element 40.

Moreover, during the pressure exertion process, the applied force-displacement variation may be obtained. When two consecutive displacement d and applied force F signals are obtained, the slope ΔF/Δx of the relationship curve between the applied force F and the displacement d may be calculated by the processing unit 40, and the point where the slope is an extreme value (maximum value or minimum value) is the characteristic critical point Pᵢ.

As shown in FIG. 5B, a relationship curve 500 is a relationship curve between the slope of the relationship curve 400 of FIG. 5A and the displacement.

According to some embodiments, the processing element 40 uses a curve fitting operation to obtain the fitting function f(x) of the relationship curve 500 of slope versus displacement. The fitting function f(x) may be a polynomial function, wherein the power of the highest order term of the polynomial function is greater than or equal to two, and the present application is not limited thereto. As shown in FIG. 5B, in the present embodiment, the relationship curve 500 is fitted with a polynomial to the sixth degree to obtain a fitting function f(x)=875.92x⁶−2400.1x⁵+2355.8x⁴−1001.7x³+154.41x²+18.241x+3.7629.

The extreme (large) value of the fitting function f(x) calculated by the processing unit 40 falls at the point 512, that is, at the point 512, the variation trend of the slope is reversed, which is the characteristic critical point Pᵢ. According to the fitting function f(x), the displacement corresponding to the characteristic critical point Pᵢ (point 512) is d=0.68 mm, and the applied force is 6.5 g. Since the pressing plane of the force-applying element 10 is circular with a diameter of 6.12 mm, after conversion, it is equivalent to a pressure of 16.25 mmHg, which is similar to the intraocular pressure of 15 mmHg set by the artificial cornea 110′.

In other embodiments, the fitting function f(x) may also be a polynomial to the second to fifth degree, and the obtained results are similar to the results of the polynomial to the sixth degree in the present embodiment.

Therefore, as shown in FIG. 5A and FIG. 5B, the relationship curve 400 between the applied force F and the displacement d is measured by the intraocular pressure detecting device 1, and the relationship curve 500 between the slope of the relationship curve 400 and the displacement is calculated, and the maximum value (that is, point 512) is found as the characteristic critical point Pᵢ with the fitting function f(x), and then the corresponding applied force F is obtained from the relationship curve 400 from the displacement d of the characteristic critical point Pᵢ to obtain the intraocular pressure value.

Since FIG. 5A and FIG. 5B obtain the characteristic critical point Pᵢ via different methods, although the results obtained are slightly different, the results obtained by both are within the allowable error range. Therefore, it may be considered that the two obtained consistent results, and both are close to the simulated set values.

In the embodiment shown in FIG. 1, since the force-applying element 10 of the intraocular pressure detecting device 1 is in direct contact with the cornea 110, discomfort often occurs to the subject during actual operation. However, in another embodiment, the intraocular pressure detecting device may apply force to the eyelid and the cornea at the same time. At this time, the subject's eye may be closed, and the intraocular pressure may be detected in such a way that the eyelid covers the cornea and the cornea is not directly in contact with the intraocular pressure detecting device.

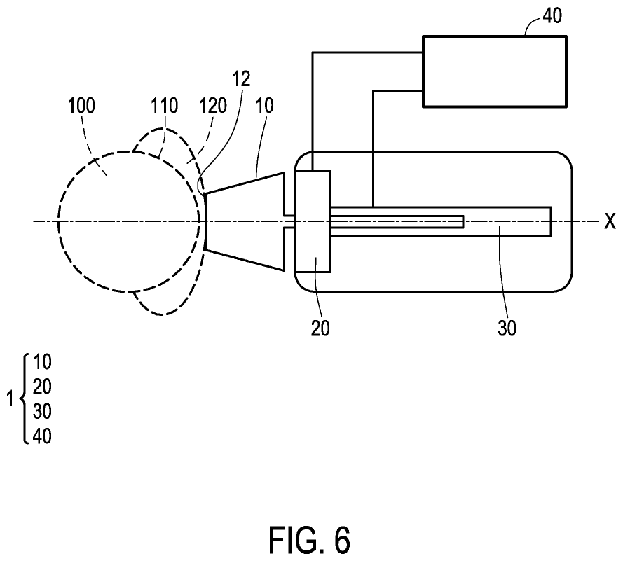

FIG. 6 is a schematic diagram of an intraocular pressure detecting device according to an embodiment of the present application. FIG. 6 is the same as the intraocular pressure detecting device 1 shown in FIG. 1, and is not repeated herein. However, it should be noted that, in FIG. 6, when the intraocular pressure detecting device 1 is in use, the force-applying element 10 is not directly in contact with the cornea 110, but is in contact with the eyelid 120 located in front of the cornea 110. Therefore, when the force-applying element 10 applies the force F, the force-applying element 10 simultaneously applies pressure to the eyelid 120 and the cornea 110.

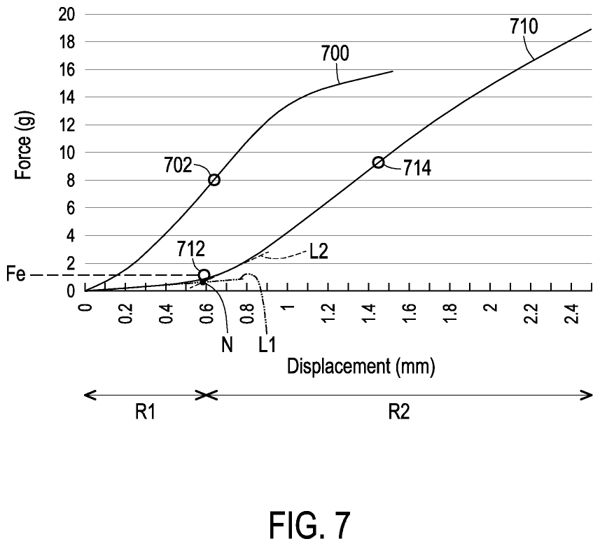

FIG. 7 is a relationship graph between applied force and displacement of an intraocular pressure detecting device according to an embodiment of the present application.

Since the cornea 110 is a tissue attached to the surface of the eyeball 100, the cornea 110 and the eyeball 100 may be considered as a whole. When pressure is applied to the cornea 110 of the eyeball 100 and the eyelid 120 at the same time, the cornea 110 (including the eyeball 100) and the eyelid 120 may be regarded as two springs with elastic coefficients k1 and k2 respectively. Therefore, the cornea 110 (including the eyeball 100) and the eyelid 120 as a whole may be regarded as a group of equivalent series springs having an elastic coefficient k.

Please refer to FIG. 7. As shown from FIG. 7, a relationship curve 700 is the relationship curve between applied force and displacement obtained by directly applying the force F to the cornea 110 by the intraocular pressure detecting device 1, and an inflection point 702 of the relationship curve 700 is obtained as the characteristic critical point Pᵢ by the method as shown in FIG. 5A. In the present embodiment, the displacement d of the characteristic critical point Pᵢ is about 0.6 mm, and the applied force F is 8 g. In other embodiments, the characteristic critical point Pᵢ may also be obtained by the method as shown in FIG. 5B, but is not limited thereto.

Moreover, a relationship curve 710 is a relationship curve between the applied force F and the displacement d when the intraocular pressure detecting device 1 simultaneously applies the force F to the cornea 110 and the eyelid 120. When the force-applying element 10 exerts pressure to the cornea 110 and the eyelid 120 of the eyeball 100 at the same time, a boundary point 712 may be defined to divide the relationship curve 710 into a first curve portion and a second curve portion. In the first curve portion, since the rigidity of the eyelid 120 is about ⅛ lower than that of the cornea 110, it is assumed that the elastic coefficient k2 of the eyelid 120 is much less than the elastic coefficient k1 of the cornea 110 (including the eyeball 100), that is, k2<<k1. Therefore, the elastic coefficient k considered as an equivalent series spring in structure is substantially equivalent to the elastic coefficient k2 of the eyelid 120, i.e., k≈k2. Therefore, in the first curve portion of the pressure exertion process, the overall displacement variation of the cornea 110 (including the eyeball 100) and the eyelid 120 is almost caused by the deformation of the eyelid 120. Therefore, the first curve portion may also be called an eyelid region R1, that is, the relationship curve 710 from the origin to the boundary point 712.

In the second curve portion of the pressure exertion process, the eyelid 120 is compressed because the eyelid 120 is subjected to force in the first stage, and the eyelid 120 at this time may be regarded as an incompressible body, and the elastic coefficient k2 of the eyelid 120 approaches infinity, that is, k2≈∞. At this time, the elastic coefficient k of the equivalent series spring is equal to the elastic coefficient k1 of the cornea 110 (including the eyeball 100), that is, k≈k1. Therefore, the second curve portion of the pressure exertion process is equivalent to exerting pressure to the cornea 110 of the eyeball 100, so the second curve portion may also be called a corneal region R2, that is, the rest of the relationship curve 710.

Comparing the relational curve 700 and the relational curve 710, it may be known that since the relational curve 710 is the relational curve of applied force and displacement obtained by applying the force F to the cornea 110 and the eyelid 120 by the intraocular pressure detecting device 1 at the same time, the variation of the curve further includes the eyelid region R1 compared to the relationship curve 700, so the variation of the relationship curve 710 in the cornea region R2 may be regarded as a shift of the relationship curve 700.

The relationship curve 710 is analyzed by the processing unit 40, and the boundary point 712 between the eyelid region R1 and the cornea region R2 may be calculated. In some embodiments, the boundary point 712 may be obtained in the following manner. As mentioned earlier, the elastic coefficient k≈k2 of the eyelid region R1, and the elastic coefficient k≈k1 of the corneal region R2. Therefore, corresponding to the variation of the relationship curve 710, it may be known that the slope of the relationship curve 710 in the eyelid region R1 should approach k2, and the slope in the cornea region R2 should approach k1. Therefore, a first tangent line L1 may be selected according to the slope of the relationship curve 710 in the eyelid region R1, and a second tangent line L2 may be selected according to the slope of the relationship curve 710 in the cornea region R2. As shown in FIG. 7, an intersection point N of the first tangent line L1 and the second tangent line L2 is calculated, and the results show that the intersection point N falls at a corresponding displacement value of about 0.58 mm, and the displacement value of 0.58 mm according to the intersection point N corresponding to the relationship curve 710 is the boundary point 712. In addition, the applied force corresponding to the boundary point 712 is Fe.

The boundary point 712 is set as the origin of the new coordinates, and the relationship curve 710 in the cornea region R2 is analyzed in the manner of pure measurement of the cornea as described in FIG. 5A or FIG. 5B to find the characteristic critical point Pᵢ, and the applied force F corresponding to the characteristic critical point Pᵢ is calculated, and then the intraocular pressure is calculated by (F−Fe)/A.

As mentioned earlier, when intraocular pressure testing is performed, the force-applying element 10 of the intraocular pressure detecting device 1 applies the force F to the cornea 110 on the eyeball 100 and the eyelid 120 covering the cornea 110 in the direction X, so that the eyelid 120 and the cornea 110 are deformed. During the measurement process, the applied force F in the direction X is sensed by the force-sensing element 20, and the displacement d in the direction X is sensed by the displacement-sensing element 30. The processing unit 40 receives the sensing results from the force-sensing unit 20 and the displacement-sensing unit 30 to obtain the relationship curve 710 between the applied force F and the displacement d. Next, the processing unit 40 further analyzes the relationship curve 710 of the applied force F and the displacement d to obtain the boundary point 712 of the relationship curve 710. The boundary point 712 divides the relationship curve 710 into a first curve portion (the eyelid region R1) and a second curve portion (the cornea region R2), and the applied force Fe corresponding to the boundary point 712 is the first applied force. Then, the boundary point 712 is set as the origin of the new coordinates, the second curve portion is analyzed to obtain the characteristic critical point Pᵢ, and the applied force F corresponding to the characteristic critical point Pᵢ is the second applied force. Lastly, the intraocular pressure is calculated according to the difference between the second applied force F and the first applied force.

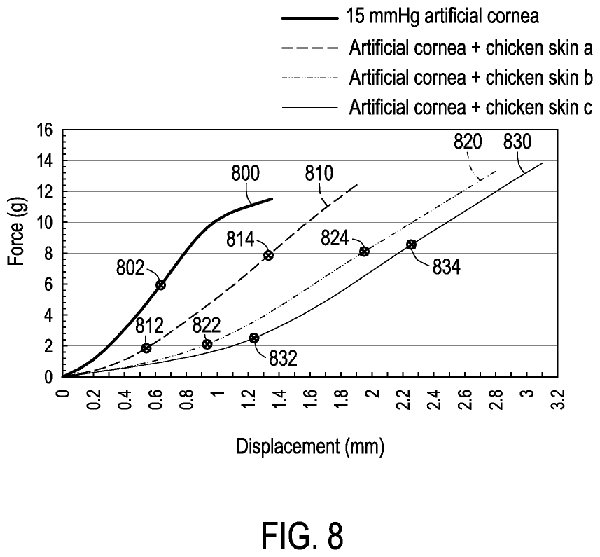

FIG. 8 is a relationship graph between applied force and displacement of an intraocular pressure detecting device according to an embodiment of the present application. Please refer to FIG. 8. In the present embodiment, the intraocular pressure test platform 2 shown in FIG. 4 is used for testing, and the experimental conditions and parameters are the same as those described above, and are not repeated herein. Note, however, that in the present embodiment, in addition, chicken skin is used to simulate the eyelid 120, and the intraocular pressure detecting device 1 measures the intraocular pressure of the combination of the artificial cornea 110′ and the chicken skin (the simulated eyelid 120) in different situations. In addition, according to the parameter calculation of the present embodiment, an intraocular pressure value of 15 mmHg is equivalent to the artificial cornea 110′ receiving an applied force F of 6 grams.

In Example 1, the intraocular pressure detecting device 1 directly measures the force F exerted on the artificial cornea 110′, and the results are shown in a relationship curve 800. An inflection point 802 of the relationship curve 800 is obtained by a method similar to that described in FIG. 5A, and the force received by the artificial cornea 110′ is 5.97 grams, which is close to the theoretical value of 6 grams.

In Example 2, the intraocular pressure detecting device 1 measures the force F simultaneously applied to a chicken skin a (simulated eyelid) and the artificial cornea 110′. During the experiment, the artificial cornea 110′ was covered with the chicken skin a having a thickness of 2 mm to 3 mm to simulate the situation that the eyelid 120 covers the cornea 110. The results are shown in a relation curve 810. A boundary point 812 between the eyelid region R1 and the cornea region R2 of the relationship curve 810 is obtained by the above method, and taking the applied force Fe=1.94 g corresponding to the boundary point 812 as the new origin, an inflection point 814 is calculated for the relationship curve 810 of the cornea region R2, and the applied force F corresponding to the inflection point 814 is obtained to be 7.97 g. According to the above results, the applied force on the artificial cornea 110′ is calculated as (F−Fe)=(7.97−1.94)=6.03 grams, which is close to the theoretical value of 6 grams.

In Example 3, the intraocular pressure detecting device 1 measures the force F simultaneously applied to a chicken skin b (simulated eyelid) and the artificial cornea 110′. During the experiment, the artificial cornea 110′ was covered with the chicken skin b having a thickness of about 2 mm to 3 mm to simulate the situation that the eyelid 120 covers the cornea 110. The results are shown in a relation curve 820. A boundary point 822 between the eyelid region R1 and the cornea region R2 of the relationship curve 820 is obtained by the above method, and taking the applied force Fe=2.18 g corresponding to the boundary point 822 as the new origin, an inflection point 824 is calculated for the relationship curve 820 of the cornea region R2, and the applied force F corresponding to the inflection point 824 is obtained to be 8.19 g. According to the above results, the applied force on the artificial cornea 110′ is calculated as (F−Fe)=(8.19−2.18)=6.01 grams, which is close to the theoretical value of 6 grams.

In Example 4, the intraocular pressure detecting device 1 measures the force F simultaneously applied to a chicken skin c (simulated eyelid) and the artificial cornea 110′. During the experiment, the artificial cornea 110′ was covered with the chicken skin c having a thickness of about 2 mm to 3 mm to simulate the situation that the eyelid 120 covers the cornea 110. The results are shown in a relation curve 830. A boundary point 832 between the eyelid region R1 and the cornea region R2 of the relationship curve 830 is obtained by the above method, and taking the applied force Fe=2.57 g corresponding to the boundary point 832 as the new origin, an inflection point 834 is calculated for the relationship curve 830 of the cornea region R2, and the applied force F corresponding to the inflection point 834 is obtained to be 8.63 g. According to the above results, the applied force on the artificial cornea 110′ is calculated as (F−Fe)=(8.63−2.57)=6.06 grams, which is close to the theoretical value of 6 grams.

The detailed measurement results are shown in Table 1.

TABLE 1
Verification results of simulated cornea having 15
mmHg intraocular pressure and different eyelids

| Unit (g) | Example 1 | Example 2 | Example 3 | Example 4 |
|---|---|---|---|---|
| Inflection point (F) | 5.97 | 7.97 | 8.19 | 8.63 |
| New origin (Fe) | | 1.94 | 2.18 | 2.57 |
| Force on cornea (F − Fe) | 5.97 | 6.03 | 6.01 | 6.06 |
| Theoretical value of | 6 | 6 | 6 | 6 |
| applied force | | | | |
| corresponding to 15 | | | | |
| mmHg intraocular | | | | |
| pressure | | | | |

Therefore, the intraocular pressure detecting device provided by the present application may calculate the intraocular pressure by measuring the relationship curve between the force applied by the force-applying element on the cornea and displacement, and calculating the characteristic critical point of the relationship curve by the calculation of the processing element.

Moreover, the intraocular pressure detecting device and the intraocular pressure measurement method provided in the present application can, in the case of directly applying force to the cornea or indirectly applying force to the cornea through the eyelids, obtain the intraocular pressure value by measuring the relationship curve between applied force and displacement and using data analysis to reduce discomfort during intraocular pressure measurement.

It will be apparent to those skilled in the art that various modifications and variations may be made to the structures of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An intraocular pressure detecting device, comprising:
a force-applying element adapted to apply a force to a target surface on a cornea of an eyeball in a direction, so that the target surface is deformed;
a force-sensing element coupled to the force-applying element and adapted to sense the force applied by the force-applying element in the direction;
a displacement-sensing element coupled to the force-applying element and adapted to sense a displacement of the force-applying element in the direction; and
a processing element electrically connected to the force-sensing element and the displacement-sensing element to obtain a relationship curve between the applied force and the displacement,
wherein the processing element analyzes the relationship curve between the applied force and the displacement to obtain a characteristic critical point, and obtains an intraocular pressure value of the eyeball according to the applied force corresponding to the characteristic critical point.

2. The intraocular pressure detecting device of claim 1, wherein the processing element obtains a fitting function of the relationship curve between the applied force and the displacement using a curve fitting operation and calculates the fitting function to obtain an inflection point of the relationship curve between the applied force and the displacement, and the characteristic critical point is the inflection point.

3. The intraocular pressure detecting device of claim 2, wherein the inflection point is a position where a second differential of the fitting function is zero.

4. The intraocular pressure detecting device of claim 2, wherein the fitting function is a polynomial function, and a power of a highest order term of the polynomial function is greater than or equal to three.

5. The intraocular pressure detecting device of claim 1, wherein the processing element calculates a slope of the relationship curve between the applied force and the displacement, obtains a fitting function of a relationship curve between the slope and the displacement using a curve fitting operation, and calculates an extreme value of the fitting function to obtain the characteristic critical point.

6. The intraocular pressure detecting device of claim 5, wherein the fitting function is a polynomial function, and a power of a highest order term of the polynomial function is greater than or equal to two.

7. An intraocular pressure detecting method, comprising:
applying a force to a target surface on a cornea of an eyeball in a direction, so that the target surface is deformed;
sensing the applied force in the direction;
sensing a displacement in the direction;
obtaining a relationship curve between the applied force and the displacement;
analyzing the relationship curve between the applied force and the displacement to obtain a characteristic critical point; and
obtaining an intraocular pressure value of the eyeball according to the applied force corresponding to the characteristic critical point.

8. The intraocular pressure detecting method of claim 7, wherein the step of analyzing the relationship curve between the applied force and the displacement to obtain the characteristic critical point further comprises:
obtaining a fitting function of the relationship curve between the applied force and the displacement using a curve fitting operation;
differentiating the fitting function to obtain an inflection point of the relationship curve between the applied force and the displacement; and
using the inflection point as the characteristic critical point.

9. The intraocular pressure detecting method of claim 8, wherein the inflection point is a position where a second differential of the fitting function is zero or a position where a first differential of the fitting function is an extreme value.

10. The intraocular pressure detecting method of claim 7, wherein the step of analyzing the relationship curve between the applied force and the displacement to obtain the characteristic critical point further comprises:
calculating a slope of the relationship curve between the applied force and the displacement;
obtaining a fitting function of a relationship curve between the slope and the displacement using a curve fitting operation; and
calculating an extreme value of the fitting function to obtain the characteristic critical point.

11. An intraocular pressure detecting method, comprising:
applying a force to a cornea on an eyeball and an eyelid covering the cornea in a direction, so that the eyelid and the cornea are deformed;
sensing the applied force in the direction;
sensing a displacement in the direction;
obtaining a relationship curve between the applied force and the displacement;
analyzing the relationship curve between the applied force and the displacement to obtain a boundary point of the relationship curve between the applied force and the displacement, wherein an applied force corresponding to the boundary point is a first applied force, the boundary point divides the relationship curve between the applied force and the displacement into a first curve portion and a second curve portion, the first curve portion is between an origin of the relationship curve between the applied force and the displacement to the boundary point, and the second curve portion is the remaining relationship curve between the applied force and the displacement;
analyzing the second curve portion to obtain a characteristic critical point, wherein the applied force corresponding to the characteristic critical point is a second applied force; and
calculating an intraocular pressure value of the eyeball according to a difference between the second applied force and the first applied force.

12. The intraocular pressure detecting method of claim 11, wherein the step of analyzing the relationship curve between the applied force and the displacement to obtain the boundary point of the relationship curve between the applied force and the displacement comprises:
selecting a first tangent line according to a slope of the first curve portion;
selecting a second tangent line according to a slope of the second curve portion;
calculating an intersection point of the first tangent line and the second tangent line, wherein the displacement corresponding to the intersection point corresponding to the relationship curve between the applied force and the displacement is the boundary point.

13. The intraocular pressure detecting method of claim 11, wherein the step of analyzing the second curve portion to obtain the characteristic critical point comprises:
obtaining a fitting function of the second curve portion using a curve fitting operation;
differentiating the fitting function to obtain an inflection point of the second curve portion; and
using the inflection point as the characteristic critical point.

14. The intraocular pressure detecting method of claim 13, wherein the inflection point is a position where a second differential of the fitting function is zero or a position where a first differential of the fitting function is an extreme value.

15. The intraocular pressure detecting method of claim 11, wherein the step of analyzing the second curve portion to obtain the characteristic critical point comprises:
calculating a slope of the second curve portion;
obtaining a fitting function of the slope and the second curve portion using a curve fitting operation; and
calculating an extreme value of the fitting function to obtain the characteristic critical point.
